(19) United States
(12) Patent Application Publication
(10) Pub. No.: US 2003/0166570 A1
(43) Pub. Date: Sep. 4, 2003

(54) Peptide and its use in the treatment of hyperpigmentation conditions

(76) Inventors: Anthony J. Thody, Shipley (GB); John M. Wood, Bradford (GB); Karin U. Schallreuter, Bradford (GB)

Correspondence Address: ERIC S. SPECTOR, JONES, TULLAR & COOPER, P.C., P.O. Box 2266, Eads Station, Arlington, VA, 22202, US

(21) Appl. No.: 09/777,656

(22) Filed: Feb. 7, 2001

(51) Int. Cl. A61K 38/08; C07K 7/06

(52) U.S. Cl. 514/16; 530/329

(57) ABSTRACT

A peptide having the formula:

X—N(H)-A¹-B²-C³-D⁴-Lys⁵-Lys⁶-Arg⁷-C(O)—Y

wherein

A, B, C and D are amino acid residues;

X is selected from the group consisting of H, a pharmaceutically acceptable amine blocking group and, together with Y, a covalent bond connecting the carbonyl group of Arg⁷ to the amine group of A¹; and

Y is selected from the group consisting of OH, a pharmaceutically acceptable carboxyl blocking group and, together with X, a covalent bond connecting the carbonyl group of Arg⁷ to the amine group of A¹. Such peptides may be used topically to treat hyperpigmentation conditions such as melanism and melasma.

### BACKGROUND TO THE INVENTION

The present invention relates to a peptide, a composition comprising a peptide and a method of treatment or prophylaxis of hyperpigmentation conditions using a composition comprising a peptide.

Hyperpigmentation is a generic term for conditions in which an abnormal build up of pigment such as melanin results in darkening of body tissue, particularly the skin. Both melanosis and melasma are examples of hyperpigmentation conditions. Hyperpigmentation, particularly in regions of the skin that are usually exposed, for example around the face and hands, can be distressing for the sufferer and may result in a lack of self-confidence and even depression.

Melanin is a black or dark brown pigment formed from the amino acid tyrosine in body cells known as melanocytes found in the epidermis. The production of melanin (“melanogenesis”) is regulated by the interaction of α-melanocyte stimulating hormone (“α-MSH”), derived from the precursor protein proopiomelanocortin (“POMC”), with the cAMP-coupled melanocortin-1 (“MC-1”) receptors found on melanocytes.

α-MSH is a polypeptide hormone having 13 amino acid residues (a 13-mer peptide) that is secreted by the anterior pituitary gland, and is expressed also in skin melanocytes and keratinocytes. Acetylated α-MSH has the following primary sequence:

Ac-Ser¹-Tyr²-Ser³-Met⁴-Glu⁵-His⁶-Phe⁷-Arg⁸-Trp⁹-Gly¹⁰-Lys¹¹-Pro¹²-Val¹³-NH₂

Adrenocorticotropin hormone (“ACTH”) is a 39-mer polypeptide hormone that is also expressed in skin melanocytes and keratinocytes. ACTH has the following primary sequence:

Ser¹-Tyr²-Ser³-Met⁴-Glu⁵-His⁶-Phe⁷-Arg⁸-Trp⁹-Gly¹⁰-Lys¹¹-Pro¹²-Val¹³-Gly¹⁴-Lys¹⁵-
Lys¹⁶-Arg¹⁷-Arg¹⁸-Pro¹⁹-Val²⁰-Lys²¹-Val²²-Tyr²³-Pro²⁴-Asn²⁵-Gly²⁶-Ala²⁷-Glu²⁸-Asp²⁹-
Glu³⁰-Ser³¹-Ala³²-Glu³³-Ala³⁴-Phe³⁵-Pro³⁶-Leu³⁷-Glu³⁸-Phe³⁹-NH₂

ACTH is derived from POMC and is related to α-MSH in that the peptide fragment defined by residues 1-13 of ACTH (“ACTH 1-13”) has the same primary sequence as desacetyl α-MSH. ACTH is present in the skin and has been shown to bind to the MC-1 receptor thereby stimulating melanogenesis.

Tsatmali et al (Annals of the New York Academy of Sciences, October 1999, vol. 885, pp. 466-469), Wakamatsu et al (Pigment Cell Research, 1997, vol. 10, pp. 288-297) and Schallreuter et al (J. Invest. Derm., March 2000, vol. 114, No. 3, pp 430-437) disclose results of comparative experiments regarding the interaction of the MC-1 receptor with α-MSH, ACTH 1-10, ACTH 1-17 and ACTH. The relative affinities of these peptides for the MC-1 receptor and the stimulation of cAMP have been determined as:

ACTH 1-17=α-MSH>ACTH

with ACTH 1-10 having no activity (in the physiologic range for signal transduction). It is stated (in Schallreuter et al, published March 2000) that the preferred activity of ACTH 1-17 and the lack of activity of ACTH 1-10 would appear to suggest that the partial sequence ACTH 11-17 is important for binding to the MC-1 receptor. As far as the present Inventors are aware, there has been no prior public disclosure of a discrete 7-mer peptide having the same primary sequence as ACTH 11-17, i.e.

Lys¹⁽¹¹⁾-Pro²⁽¹²⁾-Val³⁽¹³⁾-Gly⁴⁽¹⁴⁾-Lys⁵⁽¹⁵⁾-Lys⁶⁽¹⁶⁾-Arg⁷⁽¹⁷⁾

[position of amino acids in ACTH shown in ( )]. Previous studies of the MC-1 receptor protein show that Asp¹¹⁷ and Asp¹⁸⁴ are critical for receptor function and that Ser⁶, Glu²⁶⁹ and Thr²⁷² have a significant effect on receptor binding activity. Computer modeling docking experiments between ACTH 11-17 and the MC-1 receptor (carried out by the Inventors) show that Lys¹⁵ (of ACTH 11-17) interacts with Ser⁶ (of the receptor protein) and ion pairs with Asp¹⁷ (of the receptor protein), that Lys⁶ (of ACTH 11-17) ion pairs with Glu²⁶⁹ (of the receptor protein) and that Arg¹⁷ (of ACTH 11-17) ion pairs with Asp¹⁸⁴ (of the receptor protein) and interacts with Thr²⁷² (of the receptor protein).

Substitution of the four basic amino acid residues in ACTH 11-17 (Lys¹¹, Lys¹⁵, Lys¹⁶ and Arg¹⁷) with alanine residues reduces significantly the affinity of the 7-mer peptide with the receptor site (see Examples) and indicates that Arg¹⁷ is critical for the formation of the ACTH 11-17/MC-1 receptor complex. Results from these substitution experiments provide at least a partial explanation of the preferred affinity of ACTH 1-17 over α-MSH (i.e. ACTH 1-13) and ACTH 1-10 as binding affinity is reduced significantly with substitution of at least one of important basic residues. Without wishing to be bound by any particular theory, it is believed that synthetic 7-mer peptides having lysine residues at positions 5 and 6 and an arginine residue at position 7 bind to the MC-1 receptor and elicit an IP₃/DAG cascade within a melanocyte (see Example 1) and as they have no effect on cAMP may, in addition, act as antagonists for the binding and actions of natural POMC peptides.

There is a need for an effective treatment for hyperpigmentation that is easy to administer, preferably by the sufferers themselves, that has little or no side effect. Ideally, the treatment would involve the topical application of a composition comprising a depigmentation component. Such a composition would be easily applied and would have a local rather than a systemic effect thereby reducing the likelihood of unwanted side effects. This problem has been addressed by the present invention.

The present Inventors have discovered that 7-mer peptides having lysine residues in positions 5 and 6 and an arginine residue in position 7 (particularly, a 7-mer peptide having the same primary sequence as ACTH 11-17) are useful in the treatment of hyperpigmentation conditions due to their antagonism for the binding of natural POMC peptides to the MC-1 receptor. The inventors are unaware of any prior public disclosure of this group of discrete 7-mer peptides or of their use in the treatment of hyperpigmentation conditions.

### SUMMARY OF THE INVENTION

According to a first aspect of the present invention, there is provided a peptide having the formula:

X—N(H)-A¹-B²-C3-D⁴-Lys5-Lys⁶-Arg⁷-C(O)—Y

wherein

A, B, C and D are amino acid residues;

X is selected from the group consisting of H, a pharmaceutically acceptable amine blocking group and, together with Y, a covalent bond connecting the carbonyl group of Arg⁷ to the amine group of A¹; and

Y is selected from the group consisting of OH, a pharmaceutically acceptable carboxyl blocking group and, together with X, a covalent bond connecting the carbonyl group of Arg⁷ to the amine group of A¹.

Modeling studies have shown that a lysine residue at position 1 assists with the binding of the peptide to the MC-1 receptor. Therefore, in preferred embodiments, A¹ is lysine.

In ACTH 11-17, a proline residue is at position 12, a valine residue is at position 13 and a glycine residue is at position 14. Therefore, B² may be proline, C³ may be valine and D⁴ may be glycine.

As with all peptides, the peptide of the present invention may have a —NH₂ group at one end of the peptide chain and a —COOH group at the other end (i.e. when X=H and Y=OH). At physiological pH, there is a risk that a peptide will react with itself or other peptides. In order to reduce the risk of side reactions without adversely effecting the efficacy of the peptide, X may be any suitable amine blocking group, preferably an acetyl (or CH₃C(O)—) group and Y may be any suitable carboxyl blocking group, preferably a primary amino (or —NH₂) group.

In order for the peptide to fully bind to the MC-1 receptor, the peptide must assume a binding conformation in which the location in space of the two lysine residues in positions 5 and 6 and the arginine residue in position 7 corresponds with the location of the binding residues within the receptor. The peptide is preferably non-cyclic as this maximizes the flexibility of the peptide (and, therefore, its ability to assume the binding conformation) and, thus, improves the binding affinity of the peptide in the receptor.

The most preferred peptide has the primary sequence:

H₂N-Lys¹-Pro²-Val³-Gly⁴-Lys⁵-Lys⁶-Arg⁷-COOH

Preferably, the peptide is used in the treatment or prophylaxis of hyperpigmentation conditions selected from the group consisting of melanosis and melasma.

In a second aspect of the present invention, there is provided a therapeutic composition comprising a pharmacologically acceptable concentration of a peptide having the formula:

X—N(H)-A¹-B²-C³-D⁴-Lys⁵-Lys⁶-Arg⁷-C(O)—Y

wherein

A, B, C and D are amino acid residues;

X is selected from the group consisting of H, a pharmaceutically acceptable amine blocking group and, together with Y, a covalent bond connecting the carbonyl group of Arg⁷ to the amine group of A¹; and

Y is selected from the group consisting of OH, a pharmaceutically acceptable carboxyl blocking group and, together with X, a covalent bond connecting the carbonyl group of Arg⁷ to the amine group of A¹; and a therapeutically acceptable vehicle.

The concentration of the peptide within the composition is preferably from 10⁻⁶M to 10⁻³M.

The composition is preferably in a form suitable for topical application to the skin. A suitable topical composition is preferably in a form selected from the group consisting of cream, ointment, oil, lotion, emollient, solution, gel, foam, spray and powder with creams, ointments and lotions being particularly preferred.

In a third aspect of the present invention, there is provided a method for the treatment or prophylaxis of a hyperpigmentation condition in mammals by administration of a therapeutically effective amount of a composition comprising a peptide having the formula:

X—N(H)-A¹-B²-C³-D⁴-Lys⁵-Lys⁶-Arg⁷-C(O)—Y

wherein

A, B, C and D are amino acid residues;

X is selected from the group consisting of H, a pharmaceutically acceptable amine blocking group and, together with Y, a covalent bond connecting the carbonyl group of Arg⁷ to the amine group of A¹; and

Y is selected from the group consisting of OH, a pharmaceutically acceptable carboxyl blocking group and, together with X, a covalent bond connecting the carbonyl group of Arg⁷ to the amine group of A¹; and a therapeutically acceptable vehicle.

Suitable hyperpigmentation conditions for treatment using the present invention include melanosis and melasma. Preferably, the composition is applied topically to the skin.

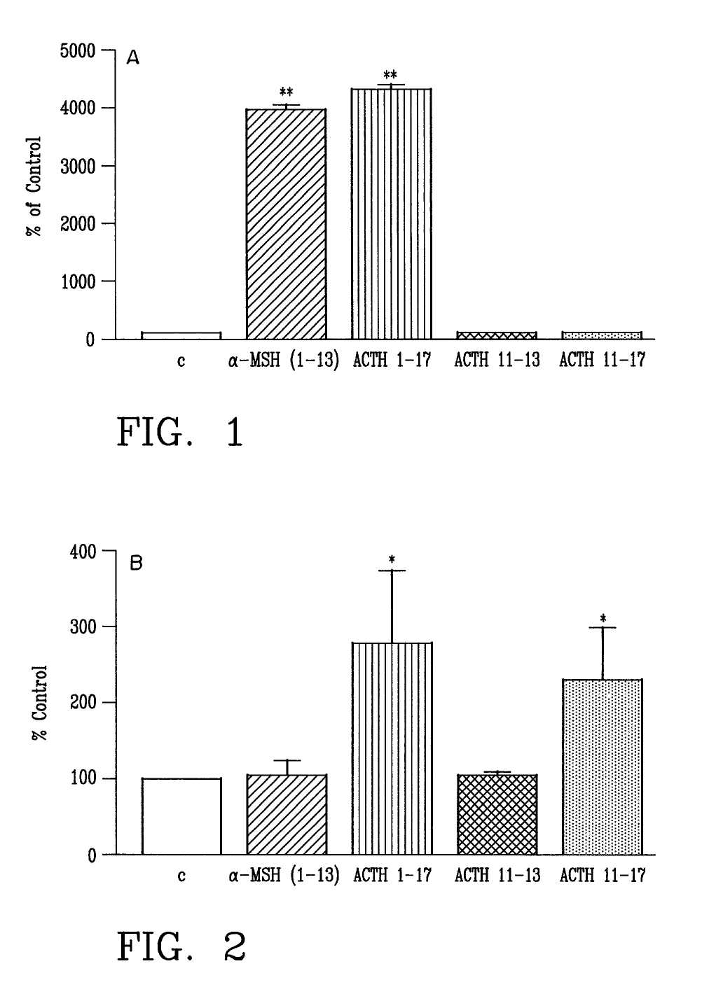

The invention will now be described by way of example only with reference to the accompanying drawings, in which: FIG. 1 is a graph depicting the results of a controlled experiment to determine the magnitude of a cAMP signal (A) provided by the interaction of α-MSH, ACTH 1-17, ACTH 11-13 and ACTH 11-17 with an MC-1 receptor; and FIG. 2 is a graph depicting the results of a controlled experiment to determine the magnitude of an IP₃/DAG signal (B) provided by the interaction of α-MSH, ACTH 1-17, ACTH 11-13 and ACTH 11-17 with an MC-1 receptor.

### EXAMPLE 1

For the measurement of cAMP, HEK 293 cells transfected with the human MC-1 receptor were seeded in 12 well plates at a density of 2×10⁵ cells/well and allowed to attach overnight. The next day, fresh media containing 1 mM IBMX was added for 30 min. Different concentrations of the POMC peptides were added for 20 min. At the end of the incubation period, cells were lysed with ice cold ethanol and the cell extracts were centrifuged and the supernatants rotary evaporated. cAMP was assayed using a commercially available kit (Nycomed Amersham plc, Amersham Place, Little Chalfont, Buckinghamshire, HP7 9NA, UK) according to the manufacturer's instructions.

### EXAMPLE 2

For the measurement of IP₃, HEK 293 cells transfected with the human MC-1 receptor were seeded in 12 well plates at a density of 2×10⁵ cells/well and allowed to attach overnight. The next day, fresh media containing 1 mM LiCl was added for 30 min. Different concentrations of the POMC peptides were added for 20 min. At the end of the incubation period, cells were lysed with 0.5 ml of ice cold 7.5% TCA. Precipitates were sedimented by centrifugation and the supernatants extracted three times with 10 volumes of water-saturated diethyl ether. Extracts were neutralized by titration to pH 7.5 with NaHCO₃. IP₃ was assayed using a commercially available kit (Nycomed Amersham pic, Amersham Place, Little Chalfont, Buckinghamshire, HP7 9NA, UK) according to the manufacturer's instructions.

### EXAMPLE 3

The following 7-mer peptides were synthesized using standard solid phase peptide synthesis techniques.

| | |
|---|---|
| H₂N-Lys.Pro.Val.Gly.Lys.Lys.Arg-COOH | Peptide I |
| H₂N-Ala.Pro.Val.Gly.Lys.Lys.Arg-COOH | Peptide II |
| H₂N-Lys.Pro.Val.Gly.Ala.Lys.Arg-COOH | Peptide III |
| H₂N-Lys.Pro.Val.Gly.Lys.Ala.Arg-COOH | Peptide IV |
| H₂N-Ala.Pro.Val.Gly.Ala.Ala.Arg-COOH | Peptide V |
| H₂N-Lys.Pro.Val.Gly.Ala.Ala.Arg-COOH | Peptide VI |

The primary sequence of Peptide I corresponds with the primary sequence for ACTH 11-17. Peptides II-VI are analogues of Peptide I in which at least one of the lysine residues has been replaced with an alanine residue.

Experiments to examine the effects of Peptides I-VI at the MC-1 receptor were carried out according to the procedures described in Examples 1 and 2.

The results of these experiments are summarized in Table 1.

TABLE 1

| | Melanocyte | | IP₃ response |
|---|---|---|---|
| Peptide | dendricity | cAMP | (HEK 293 cells) |
| I | + | − | + |
| II | − | ND | − |
| III | + | ND | + |
| IV | ND | ND | + |
| V | + | ND | + |
| VI | − | ND | − |

(ND = not done)

The results show that Peptides I, III and V stimulate melanocyte dendricity and that it would appear that this action is via the IP₃ pathway. Substitution of the lysine residues with alanine residues at positions 1, 5 and 6 (Peptides II and VI) abolish the activity of Peptide I. At present, it is not known why the substitutions at positions 5 and 6 cause a loss in activity in Peptide VI but not in Peptide V.

It will be appreciated that the invention is not restricted to the details described above with reference to the preferred embodiments but that numerous modifications and variations can be made without departing from the spirit and scope of the invention.

1. A peptide having the formula:
X—N(H)-A¹-B²-C³-D⁴-Lys⁵-Lys⁶-Arg⁷-C(O)—Y
wherein
A, B, C and D are amino acid residues;
X is selected from the group consisting of H, a pharmaceutically acceptable amine blocking group and, together with Y, a covalent bond connecting the carbonyl group of Arg⁷ to the amine group of A¹; and
Y is selected from the group consisting of oh, a pharmaceutically acceptable carboxyl blocking group and, together with X, a covalent bond connecting the carbonyl group of Arg⁷ to the amine group of A¹.

2. The peptide of claim 1 wherein A¹ is lysine.

3. The peptide of claim 1 wherein B² is proline, C³ is valine and D⁴ is glycine.

4. The peptide of claim 1 wherein X is H and Y is OH.

5. The peptide of claim 1 wherein X is Ac and Y is —NH₂.

6. The peptide of claim 1 wherein the peptide is non-cyclic.

7. The peptide of claim 1 having the primary sequence:
H₂N-Lys¹-Pro²-Val³-Gly⁴-Lys⁵-Lys⁶-Arg⁷-COOH

8. The peptide of claim 1 for use in the treatment or prophylaxis of hyperpigmentation conditions selected from the group consisting of melanosis and melasma.

9. A therapeutic composition comprising a pharmacologically acceptable concentration of a peptide having the formula:
X—N(H)-A¹-B²C³-D⁴-Lys⁵-Lys⁶-Arg⁷-C(O)—Y
wherein
A, B, C and D are amino acid residues;
X is selected from the group consisting of H, a pharmaceutically acceptable amine blocking group and, together with Y, a covalent bond connecting the carbonyl group of Arg⁷ to the amine group of A¹; and
Y is selected from the group consisting of OH, a pharmaceutically acceptable carboxyl blocking group and, together with X, a covalent bond connecting the carbonyl group of Arg⁷to the amine group of A¹; and a therapeutically acceptable vehicle.

10. The composition of claim 9 wherein the concentration of the peptide in the composition is from 10⁻⁶M to 10⁻³M.

11. The composition of claim 9 for topical application to the skin.

12. The composition of claim 11 in a form selected from the group consisting of cream, ointment, oil, lotion, emollient, gel, solution, foam, spray and powder.

13. A method for the treatment or prophylaxis of a hyperpigmentation condition in mammals by administration of a therapeutically effective amount of a composition comprising a peptide having the formula:
X—N(H-A¹-B²-C³-D⁴-Lys⁵-Lys⁶-Arg⁷-C(O)—Y
wherein
A, B, C and D are amino acid residues;
X is selected from the group consisting of H, a pharmaceutically acceptable amine blocking group and, together with Y, a covalent bond connecting the carbonyl group of Arg⁷ to the amine group of A¹; and
Y is selected from the group consisting of OH, a pharmaceutically acceptable carboxyl blocking group and, together with X, a covalent bond connecting the carbonyl group of Arg⁷ to the amine group of A¹; and a therapeutically acceptable vehicle.

14. The method of claim 13 wherein the hyperpigmentation condition is selected from the group consisting of melanosis and melasma.

15. The method of claim 13 wherein the composition is applied topically to the skin.


SEQUENCE LISTING (8 sequences)

SEQ ID NO: 1 - protein, 13 aa, Homo sapien
SYSMEHFRWGKPV

SEQ ID NO: 2 - protein, 39 aa, Homo sapien
SYSMEHFRWGKPVGKKRRPVKVYPNGAEDESAEAFPLEF

SEQ ID NO: 3 - protein, 7 aa, Homo sapien
KPVGKKR

SEQ ID NO: 4 - protein, 7 aa, Artificial Sequence
  misc_feature: Synthesized to provide a peptide of the invention
APVGKKR

SEQ ID NO: 5 - protein, 7 aa, Artificial Sequence
  misc_feature: Synthesized to provide a peptide of the invention
KPVGAKR

SEQ ID NO: 6 - protein, 7 aa, Artificial Sequence
  misc_feature: Synthesized to provide a peptide of the invention
KPVGKAR

SEQ ID NO: 7 - protein, 7 aa, Artificial Sequence
  misc_feature: Synthesized to provide a peptide of the invention
APVGAAR

SEQ ID NO: 8 - protein, 7 aa, Artificial Sequence
  misc_feature: Synthesized to provide a peptide of the invention
KPVGAAR